(19) United States
(12) Patent Application Publication
(10) Pub. No.: US 2022/0073443 A1
(43) Pub. Date: Mar. 10, 2022

(54) CANNABINOID FORMULATIONS AND METHODS OF MAKING SAME

(71) Applicant: BOTANECO INC., Calgary (CA)

(72) Inventors: Soo In YANG, Williamsville, NY (US); James SZARKO, Calgary (CA); John CHASE, Lebanon, NJ (US)

(21) Appl. No.: 17/417,061

(22) PCT Filed: Dec. 20, 2019
(86) PCT No.: PCT/CA2019/051892

§ 371 Date: Jun. 21, 2021

Related U.S. Application Data
(60) Provisional application No. 62/783,647, filed on Dec. 21, 2018.

(51) Int. Cl. C07C 39/23; C07D 311/80; C07D 311/60; A61K 9/10; A61K 47/14; A23L 33/105; A61K 36/185

(52) U.S. Cl. CPC C07C 39/23; C07D 311/80; C07D 311/60; A61K 36/185; A61K 47/14; A23L 33/105; A61K 9/10

(57) ABSTRACT

Methods for making oleosome-based cannabinoid formulations are disclosed herein. The methods include preparing and/or providing an oleosome suspension from a selected plant species, then combining the oleosome suspension with one or more cannabinoid compounds to thereby prepare a cannabinoid formulation. The cannabinoid formulations may be used to prepare various types of consumer products such as food products, personal care products, nutraceutical products, and pharmaceutical products, among others. Related compositions are also disclosed.

### CROSS-REFERENCE

This application claims the benefit of U.S. Provisional Patent Application No. 62/783,647 filed Dec. 21, 2018; the entire contents of Patent Application No. 62/783,647 are hereby incorporated by reference.

### TECHNICAL DISCLOSURE

The present disclosure generally relates to cannabinoids. More specifically, this disclosure pertains to the preparation of cannabinoid formulations and to the use thereof in the preparation of consumer products.

### BACKGROUND

Cannabinoids are a class of organic chemical compounds naturally produced by plant species belong to the Cannabaceae family including Cannabis sativa, C. indica, C. ruderalis, as well as by their hybrids.

Public interest in cannabinoids is well established because of the psychoactive effects of and the recreational use of some cannabinoids such as Δ⁹-tetrahydrocannabinol (Δ⁹-THC). It is well known that other cannabinoids such as cannabidiol, may provide desirable anti-anxiety and anti-psychotic effects.

The legislative status for Cannabis has been changing rapidly around the world with legal frameworks currently in place in various countries including Canada, The Netherlands, Germany, and several states in the United States for various personal and medicinal purposes. The recent legalization of cannabis products in numerous jurisdictions has stimulated increasing commercial interests and activities in the development of cannabinoid formulations for use in the preparation of consumer products.

One problem associated with preparing formulations comprising cannabinoid compounds is the inherently poor solubility of cannabinoid compounds in water. Δ⁹-THC, for example, is a non-crystalline compound that requires solubilization with for example, a surfactant or alternatively, its adherence to a water-miscible substance such as albumin or TWEEN® 80 (TWEEN is a registered trademark of Croda International PLC, Snaith, Great Britain). Another known approach to increasing the solubility of cannabinoid compounds in water is to chemically modify the cannabinoid structures. Thus, for example, addition of a 4-N-morpholinobutyryloxy and a cyano group to 1′,1′-dimethylpheptyl-Δ⁸-tetrahydrocannabinol is known to enhance the solubility the cannabinoid and additionally, and the resulting derivative provides some desirable pharmacological properties including interaction with cannabinoid receptors. While derivatization approaches address some of the solubility problems associated with cannabinoids, these types of approaches cause alterations to the stereochemical and pharmacological properties of cannabinoids.

Another problem associated with cannabinoid formulations is the lack of bioavailability i.e. the degree to which a cannabinoid becomes available in vivo, to a target tissue upon administration or consumption. Cannabinoid bioavailability is known to vary considerably with the route of administration. Thus, for example, the bioavailability of Δ⁹-THC upon oral administration may be much lower, for example between 4% to 12% lower, than the bioavailability of pulmonary-delivered Δ⁹-THC consumed for example, by smoking marihuana (averaging 30%). A problem associated with lower bioavailabilities is that larger doses of cannabinoids are required in order to achieve the same desired or target levels of physiological effects.

### SUMMARY

The embodiments of the present disclosure generally relate to consumer products comprising cannabinoids and to methods for making such products.

One embodiment disclosed herein, relates to selected ingredients for use in the formulation of consumer products, wherein the selected ingredients include at least cannabinoids and oleaginous ingredients in particular, plant oleosomes.

Another embodiment of the present disclosure relates to methods of making cannabinoid formulations, wherein the methods comprise:
(i) providing a cannabinoid compound;
(ii) providing an oleosome suspension comprising a plurality of oleosomes; and
(iii) combining the oleosome suspension with the cannabinoid compound to form a cannabinoid formulation, wherein the cannabinoid compound is associated with the plurality of oleosomes in the oleosome suspension.

According to one aspect, the oleosome suspension may be buffered.

According to another aspect, the cannabinoid compound may be cannabidiol (CBD) or Δ⁹-tetrahydrocannabinol (Δ⁹-THC) or cannabichromene (CBC) or mixtures thereof.

According to another aspect, the cannabinoid compound may be provided as a substantially pure compound.

According to another aspect, the cannabinoid compound may be provided as a cannabinoid suspension consisting substantially of the cannabinoid compound and a carrier fluid.

According to another aspect, the carrier fluid may be a plant oil.

According to another aspect, the carrier fluid may be a medium chain triglyceride (MCT) oil.

According to another aspect, the cannabinoid suspension may comprise from about 5% (w/w) to about 40% (w/w) of the cannabinoid compound, wherein the balance of the cannabinoid suspension comprises substantially the carrier fluid.

According to another aspect, the cannabinoid suspension may comprise about 10% (w/w) of the cannabinoid compound, the balance consisting substantially of the carrier fluid.

According to another aspect, the oleosome suspension may be a Carthamus oleosome suspension, a Prunus oleosome suspension, a Cannabis oleosome suspension, or a Brassica oleosome suspension.

According to another aspect, the cannabinoid formulation may comprise at least 90% (w/w), at least 95% (w/w), at least 96% (w/w), at least 97% (w/w), at least 98% (w/w), or at least 99% (w/w) of a mixture of an oleosome suspension, one or more cannabinoid compounds, and water.

According to another aspect, the cannabinoid formulation may comprise an oleosome suspension, one or more cannabinoid compounds suspended in a carrier fluid, and water, which together constitute at least 90% (w/w), at least 95% (w/w), at least 96% (w/w), at least 97% (w/w), at least 98% (w/w), or at least 99% (w/w) of the cannabinoid formulation.

According to another aspect, the one or more cannabinoid compounds suspended in the carrier fluid, may comprise from about 5% (w/w) to about 40% (w/w) cannabinoid compound(s) and the balance consisting substantially of the carrier fluid.

According to another aspect, the one or more cannabinoid compounds suspended in the carrier fluid, may comprise about 10% (w/w) of the cannabinoid compound and the balance consisting substantially of the carrier fluid.

Another embodiment of the present disclosure relates to methods of making consumer products comprising one or more cannabinoid compounds, wherein the method comprises:
(i) providing a cannabinoid formulation comprising one or more cannabinoid compounds and an oleosome suspension comprising a plurality of oleosomes, wherein the cannabinoid compound is associated with the plurality of oleosomes in the oleosome suspension; and
(ii) formulating the cannabinoid formulation with at least one additional ingredient suitable for the preparation of a consumer product thereby forming a consumer product.

According to one aspect, the at least one additional ingredient may be a diluent, an excipient, a carrier, or mixtures thereof.

According to another aspect, the diluent may be an aqueous solution.

According to another aspect, the consumer product may contain from about 0.1% (v/v) to about 10% (v/v) of the cannabinoid formulation.

According to another aspect, the oleosome suspension may be a buffered oleosome suspension.

According to another aspect, the oleosome suspension may be a Carthamus oleosome suspension, a Prunus oleosome suspension, a Cannabis oleosome suspension, or a Brassica oleosome suspension.

According to another aspect, the cannabinoid compound may be cannabidiol (CBD) or Δ⁹-tetrahydrocannabinol (Δ⁹-THC) or cannabichromene (CBC), or mixtures thereof.

According to another aspect, the consumer product may be a personal care product.

According to another aspect, the consumer product may be a food product.

According to another aspect, the consumer product may be a nutraceutical product.

According to another aspect, the consumer product may be a pharmaceutical product.

Another embodiment of the present disclosure relates to methods of making consumer products comprising one or more cannabinoid compounds, wherein the method comprises:
(i) providing one or more cannabinoid compounds;
(ii) preparing an oleosome suspension comprising a plurality of oleosomes;
(iii) combining the oleosome suspension with the one or more cannabinoid compounds to form a cannabinoid formulation, wherein the one or more cannabinoid compounds is/are associated with the plurality of oleosomes; and
(iv) formulating the cannabinoid formulation with at least one ingredient suitable for the preparation of a consumer product.

According to one aspect, the at least one additional ingredient may be a diluent, an excipient, a carrier, or mixtures thereof.

According to another aspect, the cannabinoid compound may be cannabidiol (CBD) or Δ⁹-tetrahydrocannabinol (Δ⁹-THC) or cannabichromene (CBC), or mixtures thereof.

According to another aspect, the consumer product comprises about 0.1% (w/w) to about 10% (w/w) of the one or more cannabinoid compounds.

According to another aspect, the consumer product may contain from about 0.1% (v/v) to about 10% (v/v) of the cannabinoid formulation.

According to another aspect, the consumer product may be a personal care product.

According to another aspect, the consumer product may be a food product.

According to another aspect, the consumer product may be a nutraceutical product.

According to another aspect, the consumer product may be a pharmaceutical product.

Another embodiment of the present disclosure relates to cannabinoid formulations, wherein the cannabinoid formulations comprise:
(i) a cannabinoid compound; and
(ii) an oleosome suspension comprising a plurality of oleosome compounds, wherein the cannabinoid compound is associated with the plurality of oleosomes.

According to one aspect, the cannabinoid formulation may comprise an oleosome suspension, a cannabinoid compound, and water, which together constitute at least 90% (w/w), at least 95% (w/w), at least 96% (w/w), at least 97% (w/w), at least 98% (w/w), or at least 99% (w/w) of the cannabinoid formulation.

According to another aspect, the cannabinoid formulation may comprise an oleosome suspension, one or more cannabinoid compounds suspended in a carrier fluid, and water, which together constitute at least 90% (w/w), at least 95% (w/w), at least 96% (w/w), at least 97% (w/w), at least 98% (w/w), or at least 99% (w/w) of the cannabinoid formulation.

According to another aspect, the carrier fluid may be a plant oil.

According to another aspect, the carrier fluid may be a medium-chain triglyceride (MCT) oil.

According to another aspect, the cannabinoid formulation may comprise the one or more cannabinoids in a range from about 5% (w/w) to about 40% (w/w) and the carrier fluid in a range from about 95% (w/w) to about 60% (w/w).

According to another aspect, the cannabinoid formulation may comprise about 10% (w/w) of the one or more cannabinoids suspended and the balance consisting substantially of the carrier fluid.

According to another aspect, the oleosome suspension may be a Carthamus oleosome suspension, a Prunus oleosome suspension, a Cannabis oleosome suspension, or a Brassica oleosome suspension.

According to another aspect, the cannabinoid compound may be cannabidiol (CBD) or Δ⁹-tetrahydrocannabinol (Δ⁹-THC) or cannabichromene (CBC), or mixtures thereof.

Another embodiment of the present disclosure relates to consumer products comprising a cannabinoid formulation, wherein the cannabinoid formulation comprises:
(i) one or more cannabinoid compounds;
(ii) an oleosome suspension comprising a plurality of oleosomes,
(iii) wherein the one or more cannabinoid compounds is/are associated with the plurality of oleosomes; and
(iv) at least one additional ingredient.

According to another aspect, the at least one additional ingredient may be a diluent, an excipient, a carrier, or mixtures thereof.

According to another aspect, the at least one additional ingredient may be an aqueous solution.

According to another aspect, the consumer product may comprise from about 0.1% (v/v) to about 10% (v/v) of the cannabinoid formulation and the balance being the at least one additional ingredient.

Another embodiment of the present disclosure relates to uses of cannabinoid formulations to make consumer products, wherein the cannabinoid formulations comprise one or more cannabinoid compounds and an oleosome suspension comprising a plurality of oleosomes, wherein the cannabinoid compound is associated with the plurality of oleosomes.

Another embodiment of the present disclosure relates to uses of cannabinoid formulations to make consumer products, wherein the cannabinoid formulations comprise one or more cannabinoid compounds and an oleosome suspension comprising a plurality of oleosomes, wherein the one or more cannabinoid compounds is/are associated with a plurality of oleosomes.

### BRIEF DESCRIPTION OF THE DRAWINGS

These and other features of the disclosure will become more apparent in the following detailed description, in which reference is made to the appended drawings, wherein:

FIGS. 1A-1C depict the chemical structures of some cannabinoid compounds, notably cannabidiol (CBD) (FIG. 1A), Δ⁹-tetrahydrocannabinol (Δ⁹-THC) (FIG. 1B), and cannabichromene (CBC) (FIG. 1C);

FIGS. 2A-2C are images of confocal Raman six spectrograph maps for the following sample materials: water (labeled “Water”), MCT (labeled “MCT”), safflower oil (labeled “Safflower”), CBD (labeled “CBD”), cover slip (labeled “cover slip”) and error score (labeled “Error”) for: a pre-associated CBD safflower oleosome formulation (FIG. 2A), a pre-associated CBD safflower oleosome formulation (FIG. 2B) and a post-add CBD safflower oleosome formulation (FIG. 2C);

FIGS. 3A-3E are charts illustrating experimental results obtained when detecting the concentration of CBD in certain sample formulations, as a function of time during which the sample formulations were stored at 45° C. Different emulsifiers were used in each of the sample formulations: safflower oleosomes (FIG. 3A), almond oleosomes (FIG. 3B), hemp oleosomes (FIG. 3C), Canola oleosomes (FIG. 3D) and polysorbate 20 (FIG. 3E). Samples were formulated with either the pre-association method or post-add method as indicated in each of the graphs;

FIG. 4 is a bar graph showing examples of color changes determined by ΔE that occurred in sample formulations during storage at 45° C. Different emulsifiers in the sample formulations were: safflower oleosomes, almond oleosomes, hemp oleosomes, Canola oleosomes, and polysorbate 20. Sample formulations were formulated with either the pre-association method or post-add method, as indicated;

FIG. 5 a bar graph showing examples of color change determined by ΔE when sample formulations were stored at 45° C. Different emulsifiers used in the sample formulations were: safflower oleosomes, almond oleosomes, hemp oleosomes, Canola oleosomes, and polysorbate 20. Samples were formulated with either the pre-association method or post-add method, as indicated; and

FIG. 6 is a bar graph showing examples of red-color change determined by Aa when sample formulations were stored under UV conditions. Different emulsifiers used in the sample formulations were: safflower oleosomes, hemp oleosomes, and polysorbate 20. Ratios of CBD:MCT oil ranged from 10:90, 25:75, and 35:65, as indicated. The sample formulations were formulated with the pre-association method.

### DETAILED DESCRIPTION OF THE DISCLOSURE

As used herein and in the claims, the singular forms, such “a”, “an” and “the” include the plural reference and vice versa unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either”.

When ranges are used herein for physical properties such as molecular weights, chemical properties, chemical formulae, and the like, all combinations and sub-combinations of ranges and specific embodiments therein are intended to be included. Other than in the operating examples or where otherwise indicated, all numbers expressing quantities of ingredients or reaction conditions used herein should be understood as modified in all instances by the term “about.” The term “about” when referring to a number or a numerical range, means that the number or the numerical range referred to is an approximation within experimental variability (or within statistical experimental error), and thus the number or the numerical range may vary between 1% and 15% of the stated number or numerical range. Furthermore, any range of values described herein is intended to specifically include the limiting values of the range, and any intermediate value or sub-range within the given range, and all such intermediate values and sub-ranges are individually and specifically disclosed (e.g. a range of 1 to 5 includes 1, 5, and all values therebetween). Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

Unless otherwise defined, scientific and technical terms used in connection with the formulations described herein shall have the meanings that are commonly understood by those of ordinary skill in the art. The terminology used herein is for the purpose of describing particular embodiments only, and is not intended to limit the scope of the present disclosure, which is defined solely by the claims.

The terms “cannabinoid” or “cannabinoid compound” may be used interchangeably herein, and refer to a class of chemical compounds capable of interacting as a ligand with human cannabinoid receptor proteins and include, in particular, the class of aromatic chemical compounds that may be formed in Cannabis, including the chemical compounds cannabigerol (CBG), cannabichromene (CBC), cannabidiol (CBD), Δ⁹-tetrahydrocannabinol (Δ⁹-THC), canabicyclol (CBL), canabielsoin (CBE), cannabinol (CBN), canabitriol (CBT), among others.

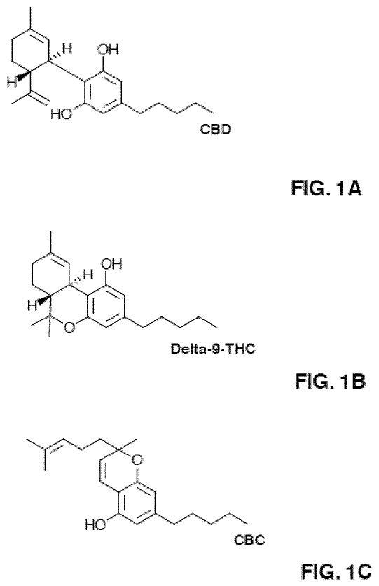

The terms “cannabidiol” and “CBD”, which may be used interchangeably herein, refer to the chemical compound set forth in FIG. 1A.

The terms “Δ⁹-tetrahydrocannabinol” and “Δ⁹-THC”, which may be used interchangeably herein, refer to the chemical compound set forth in FIG. 1B.

The terms “cannabichromene” and “CBC”, which may be used interchangeably herein, refer to the chemical compound set forth in FIG. 1C.

“Brassica” as used herein, refers to a plant belonging to the biological genus Brassica and includes, without limitation, the species Brassica napus, Brassica juncea, Brassica carinata, Brassica nigra Brassica rapa, Camelina sativa, and further includes all species, subspecies, plant cultivars, varieties, hybrids and genotypes belonging to the aforementioned genus, including the Canola variety.

“Cannabis” as used herein, refers to a plant belonging to the biological genus Cannabis and includes, without limitation, the species Cannabis sativa, Cannabis indica and Cannabis ruderalis, and further includes all species, subspecies, plant cultivars, varieties, hybrids and genotypes belonging to the aforementioned genus, including the hemp variety.

“Carthamus” as used herein, refers to a plant belonging to the biological genus Carthamus and includes, without limitation, the species Carthamus tinctorius, also known as safflower, Carthamus alexandrinus, Carthamus arborescens, Carthamus baeticus, Carthamus creticus, Carthamus flavescens, Carthamus glaucus, Carthamus lanatus, Carthamus leucocautos, Carthamus oxyacantha, Carthamus palaestinus and Carthamus turkestanicus, and further includes all species, subspecies, plant cultivars, varieties, hybrids and genotypes belonging to the aforementioned genus, including the safflower variety.

“Prunus” as used herein, refers to a plant belonging to the biological genus Prunus, including any species belonging to the subgenus Amygdalis, Prunus, Cerasus, Lithocerasus, Padus and Laurocerasus and includes, without limitation, the species Prunus dulcis, also known as almond, and further also known as Amygdalis communis, Amydalus communis, Prunus amygdalis (amygdalus), and Prunus communis, and further includes all species, subspecies, plant cultivars, varieties, hybrids and genotypes belonging to the aforementioned genus, including the almond variety.

“Oleosomes” as used herein, refer to lipid containing compartments situated within and obtainable from plant cells, comprised generally of a core of triglycerides surrounded by a monolayer of phospholipids embedded with proteins, typically oleosins, caleosins and steroleosins. Oleosomes (sometimes called “oil bodies”) of different plant species have similar compositions and structures. The properties of the mixed phospholipid-protein layer at the surface of oleosomes have been found to make them particularly stable, for example, against coalescence and thermal processing. Oleosomes serve as lipid storage sites and sources of energy during seed germination. Oleosomes may be recovered from the cells of reproductive organs of different plants including, without limitation, oleaginous seeds, nuts, and fruits using aqueous processing techniques to recover oleosome compositions therefrom.

“Consumer product” as used herein refers to merchandise for common use by humans, including a food product, a personal care, a nutraceutical product, a pharmaceutical product, and the like.

“Food product” and “food formulation” may be used interchangeably herein, and refer to edible substances for oral consumption by mammals. These terms may also refer to beverages.

“Personal care product” and “personal care formulation” may be used interchangeably herein, and refer to a finished product (composition or formulation) suitable for cleaning, cleansing, hydrating, maintaining, protecting, repairing, remediating, modifying the appearance (e.g. beautifying) at least a portion of the exterior surface area of the human body including, without limitation, the skin, hair, nails, or lips, by topical application of the product to the exterior surface.

“Nutraceutical product” and “nutraceutical formulation” may be used interchangeably herein, and refer to a dietary supplement and/or a food additive that may be an alternative to a pharmaceutical active ingredient.

“Pharmaceutical product” and “pharmaceutical formulation” may be used interchangeably herein, and refer to a finished product (composition or formulation) suitable for the diagnosis, curing, treatment, or prevention of disease, for example, by topical application of the product to the exterior surface of the skin or by oral administration.

By “formulating the cannabinoid formulation to form a consumer product” it is meant that the cannabinoid formulation is combined with at least one other ingredient including, but not limited to, a diluent, an excipient, a carrier, an emulsifier, a preservative, an antimicrobial, an antioxidant, or mixtures thereof, whereby the cannabinoid formulation and at least one other ingredient are mixed together or blended or homogenized or otherwise prepared until the consumer product is formed.

As used herein, the term “emulsifier” means an additive that helps two immiscible liquids to combine and mix. Emulsifiers referred to herein may be derived from naturally occurring sources such as plants (e.g., oleosomes produced from plants) or may be animal-derived emulsifiers or may be chemically synthesized emulsifiers.

As hereinbefore mentioned, the present disclosure relates to methods of making cannabinoid formulations. The methods of the present disclosure may be used in conjunction with a wide variety of cannabinoid compounds, including naturally occurring cannabinoid compounds obtainable from Cannabis, including Cannabis sativa and Cannabis indica, for example. Once the formulations have been constituted, the formulations may be used as ingredients to make a wide variety of consumer products.

The inventors have found, surprisingly, that when the cannabinoid compounds are formulated with oleosomes, the cannabinoid formulations may enhance the solubility of the cannabinoid compounds in aqueous media, thus facilitating the formulation of aqueous consumer products such as beverages.

Furthermore, the cannabinoid formulations of the present disclosure exhibit desirable bioavailability characteristics. Therefore, in accordance with the techniques of the present disclosure, consumer products may be prepared requiring relatively small amounts of the cannabinoid compounds, yet exhibiting high cannabinoid bioavailability.

Furthermore, the cannabinoid formulations of the present disclosure may protect the cannabinoid compounds therein, from degradation. For example, CBD may be protected against degradation or protected from oxidation by, for example, heat or ultraviolet (UV) radiation thereby limiting the potential for formation of cannabidiol breakdown products. Thus, the formulations of the present disclosure may improve the shelf life of the cannabinoid compounds as well as the consumer products formulated with the present cannabinoid formulations.

Furthermore, the cannabinoid formulations of the present disclosure may facilitate the use of lower doses of the cannabinoid compounds therein. Furthermore, the present cannabinoid formulations may facilitate delivery of the cannabinoid compounds at a controlled rate for example, when formulated to form a personal care product. This may facilitate a more evenly distributed cannabinoid associated medicinal or physiological effect, and thereby reduce the frequency of dosing.

The cannabinoid formulations of the present disclosure are compatible with a range of different other formulation ingredients for production of the consumer products including excipients, diluents, carriers, and mixtures thereof thereby facilitating the preparation of a wide range of consumer products including food products, nutraceutical products, pharmaceutical products, and personal care products, wherein the cannabinoids are homogenously distributed within the consumer products.

Accordingly, one embodiment of the present disclosure pertains to methods for preparing a cannabinoid formulation, wherein an example method comprises:
(i) providing one or more cannabinoid compounds;
(ii) providing an oleosome suspension comprising a plurality of oleosomes; and
(iii) combining the oleosome suspension with the cannabinoid compound to form a cannabinoid formulation.

According to another embodiment, another example method comprises:
(i) providing one or more cannabinoid compounds;
(ii) preparing an oleosome suspension comprising a plurality of oleosomes;
(iii) providing the prepared oleosome suspension; and
(iv) combining the oleosome suspension with the cannabinoid compound to form a cannabinoid formulation.

In one aspect of the present disclosure, the cannabinoid compounds may be associated with the plurality of oleosomes in the oleosome suspension. Thus, the cannabinoid compounds may be associated with the core of triglycerides and/or the monolayer of phospholipids and/or the outer layer of protein constituents such as oleosins, caleosins, and/or steroleosins therein.

The cannabinoid compounds may be provided as a more or less pure compounds including, for example, as an at least about 95%, 96%, 97%, 98%, 99%, 99.5% or 99.9% pure cannabinoid compound in a crystalline form or an oil form.

According to some embodiments, the cannabinoid compounds may be provided as a suspension in a carrier fluid. The carrier fluid may be, for example, a plant oil, such as safflower oil, or a medium chain triglyceride oil (MCT oil) having 6-12 carbon atoms (e.g., caproic acid (C6), caprylic acid (C8), capric acid (C10), or lauric acid (C12), or mixtures thereof). The cannabinoid suspension may be prepared for example, by dissolving from about 5% (w/w) to about 40% (w/w), or at least about 95%, 96%, 97%, 98%, 99%, 99.5% or 99.9% one or more pure cannabinoid compounds in about 60% (w/w) to about 95% (w/w) of the carrier fluid.

Thus, in some embodiments, the cannabinoid compound suspended in the carrier fluid comprises from about 5% (w/w) to about 40% (w/w); or about 5% (w/w); or about 10% (w/w); or about 15% (w/w); or about 20% (w/w); about 25% (w/w); or about 30% (w/w); or about 35% (w/w), or about 40% (w/w), wherein the balance in each instance consists substantially of the carrier fluid. For example, if a 10% (w/w) of a cannabinoid compound is selected, the cannabinoid compound is suspended in 90% (w/w) the carrier fluid.

The carrier fluid is preferably highly pure, for example, at least about 99.0% pure, at least about 99.5% pure, or at least about 99.9% pure. Thus, the one or more cannabinoid compounds suspended in the carrier fluid may be a cannabinoid suspension substantially consisting of the one or more cannabinoid compounds and the carrier fluid.

According to one aspect of the present disclosure, one or more cannabinoid compounds are provided or obtained. The one or more cannabinoid compounds may be selected and used in the methods and compositions described herein. Cannabinoid compounds may be generally classified into various groups based upon their chemical structure, including CBG, CBC, CBD, Δ⁹-THC, CBL, CBE, CBN, CBT, among others, all of which may be used in accordance with the present disclosure. The one or more cannabinoid compounds may be extracted from cannabinoid-producing plants and then further processed into purified forms. Alternatively, the one or more cannabinoid compounds may be obtained by chemical synthesis using methods known to those skilled in these arts. Furthermore, it is within the scope of the present disclosure for the naturally occurring purified cannabinoid compounds and for chemically synthesized cannabinoid compounds to be further chemically derivatized to produce derivatized cannabinoid compounds for use in the methods and with the personal care products disclosed herein.

According to another aspect of the present disclosure, the one or more cannabinoid compounds may be CBD.

According to another aspect, the one or more cannabinoid compounds may be Δ⁹-THC.

According to another aspect, the one or more cannabinoid compounds may be CBC.

According to another aspect, the cannabinoid compounds may be mixtures or two or more of CBD, Δ⁹-THC, and CBC.

According to another aspect of the present disclosure, the oleosome suspension may be prepared from plants belonging to the genus Carthamus, including, without limitation, plants belonging to the plant species Carthamus tinctorius.

According to another aspect, the oleosome suspension may be prepared from plants belonging to the genus Prunus, including, without limitation, plants belonging to the plant species Prunus dulcis.

According to another aspect, the oleosome suspension may be prepared from plants belonging to the genus Brassica.

According to another aspect, the oleosome suspension may be prepared from plants belonging to the genus Cannabis.

In some embodiments, plants or plant materials such as seeds, nuts, or fruits may be obtained and used as source materials wherefrom the oleosome suspensions may be extracted. A wide variety of extraction processes and techniques may be used provided that such extraction processes comprise conditions sufficiently gentle and mild so that the oleosome structures are not disrupted or otherwise destroyed. Thus, process techniques involving organic extractants such as hexane and the like, and/or high temperatures are less suitable.

Suitable non-destructive extraction techniques include processes comprising (i) a comminution step using for example, stirring, milling, or grinding equipment and processes to disrupt plant tissues under conditions that do not substantially disrupt the integrity of the oleosomes, and (ii) one or more aqueous extraction steps comprising mixing comminuted plant material with, for example, water or a suitable aqueous buffer, and (iii) separation of the oleosome fraction from (a) insoluble plant materials such as seed hulls and cell wall materials, and (b) plant material soluble in an aqueous phase for example water-soluble plant proteins, simple sugars, and water-soluble polysaccharides. Suitable separation equipment for step (iii) include centrifugation equipment and the like.

The low-density fraction thus obtained from the separation step (iii) comprises an oleosome suspension which may be a substantially pure oleosome suspension substantially free of other plant constituents. For example, an aqueous oleosome suspension may comprise 10% (w/w) or less, 5% (w/w) or less, 4% (w/w) or less, 3% (w/w) or less, 2% (w/w) or less, 1% (w/w) or less of non-oleosome plant constituents. The recovered oleosome suspension may further be described as an oil-in-water (O/W) emulsion. The oleosome suspension may in some embodiments, comprise small quantities generally constituting 5% (w/w) or less, of ingredients selected to protect the plurality of oleosomes against undesirable alterations by physical, chemical or biological agents. Such selected ingredients may protect the oleosome suspension during long-term storage or in storage conditions wherein the ambient temperatures fluctuate. Thus, for example, small quantities of preservatives and/or agents selected to enhance the physico-chemical or sensory properties of the plurality of oleosomes may be included in the oleosome suspension. Suitable stabilizing agents may be selected for inclusion in the selected consumer products, such as a food product or personal care product. The oleosome suspension may further contain varying amounts of water for example, more than 10% and less than 65% water by volume, more preferably more than 15% and less than 50% water by volume, and most preferably more than 20% water by volume and less than 50% water by volume. The oleosome suspension may also contain minor amounts of salts. Various suitable methods for the preparation of oleosome suspensions are known in the art and are further described in for example, U.S. Pat. Nos. 6,146,645; 6,183,762; 6,210,742; 6,372,234; 6,582,710; 6,596,287; 6,599,513; 6,761,914; and 8,597,694.

In some embodiments, a buffered oleosome suspension may be prepared wherein the suspension is buffered to a pH selected from a range of about pH 3 to about pH 10 and therebetween. In order to prepare the buffered oleosome suspension in accordance herewith, in one embodiment the oleosome suspension may be combined and mixed with any buffering agent capable of buffering the oleosome suspension to obtain the oleosome suspension buffered within a pH range between about pH 3 to about pH 10 and therebetween. Thus, in some aspects, a weak acid may be used such as a sufficient quantity of citric acid, acetic acid, succinic acid, and the like, and the oleosome suspension and the weak acid are mixed together to produce the buffered oleosome suspension. In some aspects, base may be included in the buffered suspension. Suitable bases include sodium hydroxide, potassium hydroxide, and the like. In some aspects, the pH may be adjusted to a pH selected from the range of about pH 3 to about pH 10. In such buffered oleosome suspensions, a concentration [Hi] of about 10⁻³, and 10⁻¹⁰ moles per liter will be provided.

In accordance some embodiments disclosed herein, the plurality of the oleosomes in the oleosome suspension or in the buffered oleosome suspension, may be combined with one or more cannabinoid compounds and mixed, homogenized, or otherwise prepared to form a cannabinoid formulation wherein the cannabinoid compound is associated with the plurality of oleosomes in the oleosome suspension.

The operational conditions and techniques comprising the present methods may vary, but generally involve gentle mixing or stirring of the oleosome suspension at temperatures ranging from about 4° C. about 60° C. or alternatively, from about ambient room temperature to about 50° C. However, due to the relative instability of cannabinoid compounds and the related potential for formation of cannabinoid derivatives, temperatures no higher than ambient room temperature are generally preferred. Thus, for example, the cannabinoid compound may be mixed with the oleosome suspension at an ambient room temperature of about 22±5° C.

Thereafter, in some embodiments, the present cannabinoid formulations may be stored at a temperature from a range of about 4° C. to about 60° C. for at least 1 day, at least 2 days, at least 3 days, at least 4 days, at least 5 days, at least 6 days, at least 7 days, inclusive, or for a period of 1 to 2 weeks to thereby allow the formulation to equilibrate.

The concentration of the one or more cannabinoid compounds in the present cannabinoid formulations may vary, but generally constitute no more than about 35% (w/w), no more than about 20% (w/w), no more than about 15% (w/w), no more than about 10% (w/w), no more than about 5% (w/w), no more than about 3% (w/w), or no more than about 2% (w/w) of the cannabinoid formulations. Furthermore, the concentration of the one or more cannabinoid compounds in the cannabinoid formulation may be optimized or adjusted for example, by preparing a plurality of sample formulations each provided with a different concentration of a selected cannabinoid, evaluating the formulations with reference to each formulation's suitability as a personal care product in regard to providing physiological responses to a user of the product, and then selecting one or more cannabinoids provided at a selected concentration to provide most desirable physiological effects.

In some embodiments, a cannabinoid formulation prepared by the methods disclosed herein may comprise an oleosome suspension, and one or more cannabinoid compounds which together, constitute at least 90% (w/w), at least 95% (w/w), at least 96% (w/w), at least 97% (w/w), at least 98% (w/w), or at least 99% (w/w) of the cannabinoid formulation, and wherein the one or more cannabinoid compounds are associated with a plurality of oleosomes in the oleosome suspension. As hereinbefore noted, in some embodiments, the cannabinoid formulation may include small amounts of additional ingredients, such as preservative agents, antimicrobial agents, antioxidants, and the like.

In some embodiments, a cannabinoid formulation prepared by the methods disclosed herein may comprise an oleosome suspension, one or more cannabinoid compounds suspended in a carrier fluid, and optionally water, which together constitute at least 90% (w/w), at least 95% (w/w), at least 96% (w/w), at least 97% (w/w), at least 98% (w/w), or at least 99% (w/w) of the cannabinoid formulation.

In some embodiments, the one or more cannabinoid compounds suspended in the carrier fluid may comprise from about 5% (w/w) to about 40% (w/w), for about 5% (w/w); or about 10% (w/w); or about 15% (w/w); or about 20% (w/w); or about 25% (w/w); or about 30% (w/w); or about 35% (w/w), or about 40% (w/w), and wherein the balance substantially comprises the carrier fluid.

As hereinbefore noted, in some embodiments, the cannabinoid formulations disclosed herein may comprise one or more ingredients that may provide protection against degradation of the cannabinoid compound(s) therein, thus rendering the formulation suitable for long-term storage. In this respect, in some embodiments, the cannabinoid formulations disclosed herein may be stored at about 45° C. for a period of 2 months wherein during such storage time, at least about 90% (w/w), at least about 95% (w/w), or at least about 99% (w/w) of the cannabinoid compound(s) within the formulation remain chemically unaltered, i.e. the cannabinoid compound is not degraded into chemical breakdown products. In other embodiments, the cannabinoid formulations may be stored at ambient room temperatures for a period of at least about 1 year, at least about 1.5 years, or at least about 2 years, wherein during such storage at least 90% (w/w), at least 95% (w/w), or at least 99% (w/w) of the cannabinoid compound within the formulations remains chemically unaltered.

Another embodiment of the present disclosure relates to a cannabinoid formulation wherein the cannabinoid formulations comprises:
(i) one or more cannabinoid compounds; and
(ii) an oleosome suspension comprising a plurality of oleosomes, wherein the one or more cannabinoid compounds are associated with the plurality of oleosomes in the oleosome suspension.

The cannabinoid formulation of the present disclosure is further characterized by comprising an oleosome suspension containing a plurality of oleosomes wherein the one or more cannabinoid compounds are associated with the plurality of oleosomes, and thus, are generally associated with the lipid core of the oleosomes and/or the phospholipid membranes and/or the proteins associated with the phospholipid membrane.

In accordance with one aspect of the present disclosure, the cannabinoid formulation may be used to prepare a consumer product.

Accordingly, the present disclosure pertains in at least one embodiment, to a method of preparing a consumer product comprising one or more cannabinoid compounds wherein the method comprises:
(i) providing a cannabinoid formulation comprising one or more cannabinoid compounds and an oleosome suspension comprising a plurality of oleosomes, wherein the cannabinoid compound is associated with the plurality of oleosomes in the oleosome suspension; and
(ii) formulating the cannabinoid formulation with at least one selected ingredient suitable for the preparation of the consumer product to thereby form the consumer product comprising one or more cannabinoid compounds.

Another embodiment of the present disclosure relates to a method of preparing a consumer product, the method comprising:
(i) providing one or more cannabinoid compounds;
(ii) preparing an oleosome suspension comprising a plurality of oleosomes;
(iii) combining the oleosome suspension with the one or more cannabinoid compounds to form a cannabinoid formulation, wherein the one or more cannabinoid compounds associated with the plurality of oleosomes in the oleosome suspension; and
(iv) formulating the cannabinoid formulation with at least one suitable ingredient selected for the preparation of the comprising the one or more cannabinoid compounds.

Another embodiment of present disclosure relates to a use of a cannabinoid formulation comprising or consisting of one or more cannabinoid compounds and an oleosome suspension comprising a plurality of oleosomes, wherein the one or more cannabinoid compounds are associated with the plurality of oleosomes in the oleosome suspension.

According to one aspect, the cannabinoid formulation is combined with at least one other suitable ingredient selected for use in the consumer product, wherein the selected ingredient is a diluent, an excipient, a carrier, or mixtures thereof. The cannabinoid formulation and the selected ingredient are mixed or blended or homogenized, or otherwise processed until a homogenous mixture is formed, said homogenous mixture suitable for use as the consumer product. The selected diluent and/or carrier and/or excipient and or mixtures thereof may be provided in any form including, for example, a solution, a suspension, a gel, a liquid, a solid, a powder, or a crystal. The quantity of the selected ingredient may vary depending on the type of consumer product being prepared.

According to another aspect, the at least one additional ingredient may be a diluent wherein the diluent may be an aqueous solution. It is noted that in this respect, the present cannabinoid formulation may be dissolved into the aqueous solution thereby dispersing the one or more cannabinoid compounds throughout the aqueous solution.

According to another aspect, a plurality of selected other ingredients may be provided, for example at least 2, 3, 4, 5, 6, 7, 8, 9, 10 or more suitable ingredients may be combined with the cannabinoid formulation to prepare a selected consumer product. Examples of other suitable ingredients include preservative agents, antimicrobial agents, antioxidants, and the like. In embodiments hereof that include a plurality of selected suitable ingredients, such ingredients may be combined sequentially or simultaneously.

In some embodiments, a formulation suitable for inclusion in the consumer product comprising a mixture of ingredients, is pre-formed and the cannabinoid formulation may be separately provided and combined with the pre-formed ingredient mixture.

In some embodiments, the cannabinoid formulation may be incorporated during formulation. In such embodiments, the cannabinoid formulation may be added separately or alternatively, the cannabinoid formulation may be incorporated together with one or more other selected ingredients.

The manner of incorporation of the cannabinoid formulation in the consumer product may vary. In some embodiments, the cannabinoid formulation may be more or less homogenously distributed within and throughout the consumer product. For example, the cannabinoid formulation may be dispersed in an aqueous solution to form a more or less homogenous suspension may be formulated such as a beverage.

The final concentration of the cannabinoid formulation in a consumer product may vary. In general, the amount of the cannabinoid formulation may be selected to provide a suitable bioavailability of the cannabinoid compound. With the term “suitable bioavailability” it is meant that an amount of the cannabinoid formulation is selected that results in a desirable psychoactive or other physiological effect. As will be clear to those of skill in this art, the amount of the cannabinoid formulation provided may vary depending on the consumer product. Thus, there may be differences in bioavailability of the one or more cannabinoids in food products or personal care products or nutraceutical produces or pharmaceutical products comprising the cannabinoid formulation based on the selected route of administration i.e., oral administration or dermal administration, varies. In some embodiments, the cannabinoid formulation may comprise 0.1% (w/w) or about 0.1% (w/w) of the consumer product. In other embodiments, the cannabinoid formulation may comprise about 0.5% (w/w), about 1% (w/w), about 2% (w/w), about 5% (w/w), about 10% (w/w), about 20% (w/w), about 30% (w/w). In some embodiments, the cannabinoid formulation may be selected such that the amount of the cannabinoid compound in the consumer product such as a food formulation may be from about 5 mg to about 50 mg per serving of the food formulation, or from about 10 mg to about 25 mg per serving of the food formulation, or about 15 mg per serving of the food formulation.

In some embodiments, the cannabinoid formulation may be selected such that the amount of the cannabinoid compound in the consumer product is at least 0.3% (w/w), at least 0.4% (w/w), at least 0.5% (w/w), at least 0.6% (w/w), at least 0.7% (w/w), at least 0.8% (w/w), at least 0.9% (w/w), at least 1.0% (w/w), 1.5% (w/w), at least 2.0% (w/w), at least 2.5% (w/w), at least 3.0% (w/w), or from 0.3% or about 0.3% (w/w) to 3.0% (w/w) or about 3.0% (w/w). Furthermore, the concentration of the one or more cannabinoid compounds in the cannabinoid formulation may be optimized or adjusted for example, by preparing a plurality of sample formulations each provided with a different concentration of a selected cannabinoid, evaluating the formulations with reference to each formulation's suitability as a personal care product in regard to providing physiological responses to a user of the product, and then selecting one or more cannabinoids provided at a selected concentration to provide most desirable physiological effects.

In accordance with the foregoing, the present disclosure further provides, in at least one embodiment, a consumer product comprising a cannabinoid formulation wherein the cannabinoid formulation comprises:
(i) one or more cannabinoid compounds;
(ii) an oleosome suspension comprising a plurality of oleosomes, wherein the one or more cannabinoid compounds are associated with the plurality of oleosomes in the oleosome suspension; and
(iii) at least one additional selected ingredient.

In accordance herewith, the consumer product may comprise a diluent, a carrier, an excipient, or mixtures thereof that are suitable for formulating the consumer product. In some embodiments, the diluent, carrier, excipient, or mixtures thereof incorporated into the personal care products of disclosed herein, may be natural ingredients. In some embodiments, the diluent, carrier, excipient, or mixtures thereof incorporated into the personal care products of disclosed herein, may be synthetic ingredients. In some embodiments, the consumer product may additionally comprise one or more of preservative agents, antimicrobial agents, antioxidants, and the like.

A wide variety of additional ingredients may be selected to formulate the consumer product disclosed herein, depending on the type of desired consumer product, which in some embodiments may, for example, be a personal care product, a pharmaceutical product, a nutraceutical product, or a food product. It is to be noted that the mode of delivery of the cannabinoid compound to a user may vary with the type of consumer product formulated. In general, oral delivery of the cannabinoid compound will be considered and affected when food products are formulated, while dermal delivery of the cannabinoid compound will be considered and affected when personal care products or pharmaceutical products are formulated. Some example optional additional ingredients in relation to personal care products, pharmaceutical products, nutraceutical products, and food products are described below. It should be noted, however, that other ingredients may be selected as desired or appropriate, and used to prepare the consumer product comprising the cannabinoid formulation.

In some embodiments, additional ingredients that may be incorporated into the present consumer products include, for example, pH-modulating agents including NaOH, KOH, acetic acid, citric acid, and the like; surface-active agents such as cationic surfactants, anionic surfactants, zwitterionic surfactants, and the like; moisturizers such as glycerin, propylene glycol, and the like; emollients such as oils, lipids and the like; viscosity-modulating agents such as xanthan gum, carrageenan gum, and the like; and chelating agents such as EDTA and the like.

The final concentrations of the additional selected ingredients added into the present consumer products, the pHs of the present consumer products, the viscosities of the present consumer products, and other chemical and physicochemical properties of the present consumer products disclosed herein may vary substantially depending on the desired use and performance characteristics of the consumer products. Those of skill in the art will be familiar with a variety of different methodologies and techniques such as for example, heating methodologies, stirring techniques, mixing techniques, pH-adjustment techniques, viscosity-adjustment methodologies, and the like, all of which may be used, adjusted and/or optimized in to prepare selected types of consumer products.

The cannabinoid formulation in accordance with the present disclosure may be used to prepare consumer products intended for recreational use.

In addition to providing benefits as a recreational consumer product, the consumer products prepared in accordance with the methods disclosed herein, in some embodiments may provide a wide variety of different uses including, in the case of a personal care product or a pharmaceutical product a specific treatment of the exterior of the human body. In some embodiments, a personal care product disclosed herein may be a skin-care formulation such as a skin cream, a sunscreen formulation, a bath and body formulation, a hair care formulation, and the like. In the case of a food product, the present food product may additionally provide nourishment. In some embodiments, a food product in accordance with the present disclosure may be a baked good, a confection, or a beverage. In some embodiments, a pharmaceutical product prepared in accordance with the present disclosure may be an oral formulation or a topical formulation.

The above disclosure generally describes various aspects of methods and compositions of the present disclosure. Changes in form and substitution of equivalents are contemplated as circumstances might suggest or render expedient. Although specific terms have been employed herein, such terms are intended in a descriptive sense and not for purposes of limitation.

### EXAMPLES

Hereinafter are provided examples of further specific embodiments for practicing the methods of the present disclosure and also, specific embodiments pertaining to compositions of the present disclosure. The examples are provided for illustrative purposes only, and are not intended to limit the scope of the present disclosure in any way.

As used herein, the term “ΔE” refers to a measure of change in visual perception of two colors that allows for quantified determination of the differences between two colors within a color space.

As used herein, the term “Aa” refers to a measure of change between two samples of in terms of red and green tones. A higher positive Aa value indicates the sample is more “reddish” in color. A lower negative Aa value indicates the sample is more “greenish” in color.

As used herein, the terms “pre-associate” and “pre-association” may be used interchangeably herein, and refer to methods and/or techniques for combining an oil-solubilized cannabinoid compound with an oleosome suspension thereby forming a cannabinoid formulation wherein the cannabinoid compound is associated with oleosomes in the oleosome suspension, whereby the cannabinoid formulation is suitable for use as an ingredient for preparation of consumer products.

As used herein, the terms “post-add”, “post-associate”, and “post-association” may be used interchangeably herein, and refer to preparation methods or techniques wherein a first mixture is prepared by combining an oil-solubilized one or more cannabinoid compounds with at least one other ingredient, and then combining the first mixture with an oleosome suspension to produce a second mixture (a first cannabinoid-based formulation) suitable for use in formulating a consumer product. Alternatively, the terms post-add”, “post-associate”, and “post-association” may be used to refer to preparation methods or techniques wherein a third mixture is prepared by combining an oleosome suspension with at least one other ingredient, and then combining the third mixture with an oil-solubilized one or more cannabinoids to produce a fourth mixture (a second cannabinoid-based formulation) suitable for use in formulating a consumer product.

### Example 1: Preparation of Cannabinoid Formulations

A first cannabinoid formulation was prepared using a method to pre-associate a selected CBD isolate with medium-chain triglyceride oil (MCT) to solubilize the CBD isolate therein (white, crystalline powder, 98.2% pure; Mile High Labs, Broomfield, Colo., USA). The ingredient amounts used to prepare the first formulation are shown in Table 1.

TABLE 1

| | | Amount |
|---|---|---|
| Phase | Ingredient | (g) |
| 1 | CBD Isolate | 1.00 |
| 1 | Caprylic/Capric Triglyceride (MCT) | 9.00 |
| 2 | Emulsifier | 10.00 |
| | A: Safflower oleosomes (65%) | |
| | B: Almond oleosomes (60%) | 10.00 |
| | C: Hemp oleosomes (70%) | 10.00 |
| | D: Canola oleosomes (52%) | 10.00 |
| | E: Polysorbate 20 (100%) | 1.00 |

Phase 1 ingredients were premixed in a 100-mL PYREX® beaker (PYREX is a registered trademark of the Corning Glass Works Corp., Corning, N.Y., USA) using a medium speed propeller stirring (300 rpm) for approximately 20-30 minutes until the CBD was fully dissolved. Premixed Phase 1 was added to Phase 2 in a 400-mL PYREX® beaker, and then mixed with a propeller mixer at 400 rpm for 20-30 minutes, thereby producing the first cannabinoid formulation.

A second cannabinoid formulation was prepared using a method to pre-associate a CBD isolate (same as in Example 1) with safflower oil to solubilize therein the CBD isolate. The ingredient amounts used to prepare the second formulation are shown in Table 2.

Phase 1 ingredients were premixed in a 100-mL PYREX® beaker using a medium-speed propeller stirring at 300 rpm for approximately 20-30 minutes until the CBD was fully dissolved. Premixed Phase 1 was added to Phase 2 in a 400-mL PYREX® beaker, then mixed with a propeller mixer at 400 rpm for 20-30 minutes, thereby producing the second cannabinoid formulation.

TABLE 2

| | | Amount |
|---|---|---|
| Phase | Ingredient | (g) |
| 1 | CBD Isolate | 1.00 |
| 1 | Carthamus tinctorius oil (safflower) | 9.00 |
| 2 | Emulsifier | |
| | A: Safflower oleosomes (65%) | 10.00 |
| | B: Almond oleosomes (60%) | 10.00 |
| | C: Hemp oleosomes (70%) | 10.00 |
| | D: Canola oleosomes (52%) | 10.00 |
| | E: Polysorbate 20 (100%) | 1.00 |

### Example 2: Preparation of a Cannabinoid-Based Formulation Using a Post-Add Formulation Method Using MCT to Solubilize the CBD Isolate

A first cannabinoid-based formulation was prepared using a post-add method wherein a CBD isolate (the same as in Example 1) was solubilized into a medium-chain triglyceride oil (MCT). The ingredient amounts used are shown in Table 3.

TABLE 3

| Phase | Ingredient | Percentage |
|---|---|---|
| 1 | DI water | 77.73 or 86.73 |
| 1 | SEPIMAX ZEN ® | 1.25 |
| 2 | Emulsifier | 10.00 |
| | A: Safflower oleosomes 65% | 10.00 |
| | B: Almond oleosomes 60% | 10.00 |
| | C: Hemp oleosomes 70% | 10.00 |
| | D: Canola oleosomes 52% | 10.00 |
| | E: Polysorbate 20 100% | 1.00 |
| 3 | Sodium Hydroxide Solution (18%) | 0.02 |
| 4 | EUXYL ® PE9010 | 1.00 |
| 5 | CBD Isolate | 1.00 |
| 5 | Caprylic/Capric Triglyceride (MCT) | 9.00 |
| | TOTAL | 100.00 |

Phase 1 ingredients were mixed by slowly dispersing SEPIMAX ZEN® (SEPIMAX ZEN is a registered trademark of SEPPIC Corporation France, Paris, FR) into de-ionized (DI) water in a 400-mL PYREX® beaker stirred with a medium-speed propeller at 600 rpm until the suspension was homogeneous.

Then, the Phase 2 emulsifier was slowly added to the 400-mL PYREX® beaker with continued propeller mixing (400 rpm) until the suspension was homogeneous. The Phase 3 ingredient was then added under continued constant mixing to adjust the pH of the mixture to between 5.5 and 6.0.

Then, the Phase 4 ingredient, EUXYL® PE9010, a broad-spectrum preservative (EUXYL is a registered trademark of Schulke & Mayr GmbH, Norderstedt, DE) was added to the 400-mL PYREX® beaker under continued constant mixing.

The Phase 5 ingredients were premixed in a 100-mL PYREX® beaker with a medium-speed propeller stirring at 300 rpm for approximately 20-30 minutes until the CBD was fully dissolved thereby forming a cannabinoid suspension in a caprylic/capric triglyceride (MCT oil) carrier fluid.

The Phase 5 suspension was then added to the 400-mL PYREX® beaker under constant mixing thereby producing the first cannabinoid-based formulation.

### Example 3: Preparation of a Cannabinoid Formulation Using a Method to Pre-Associate an Emulsifier with a CBD Isolate and Using a MCT Oil to Solubilize the CBD Isolate

A third cannabinoid formulation was prepared using a pre-association method using the ingredients and ingredient percentages shown in Table 4.

TABLE 4

| Phase | Ingredient | Percentage |
|---|---|---|
| 1 | DI water | 77.73 or 86.73 |
| | SEPIMAX ZEN ® | 1.25 |
| 2 | CBD Isolate | 1.00 |
| | MCT oil | 9.00 |
| 3 | Emulsifier | |
| | A: Safflower oleosomes 65% | 10.00 |
| | B: Almond oleosomes 60% | 10.00 |
| | C: Hemp oleosomes 70% | 10.00 |
| | D: Canola oleosomes 52% | 10.00 |
| | E: Polysorbate 20 100% | 1.00 |
| 4 | Sodium Hydroxide Solution (18%) | 0.02 |
| 5 | EUXYL ® PE9010 | 1.00 |
| | TOTAL | 100.00 |

Phase I ingredients were mixed by slowly dispersing SEPIMAX ZEN® in DI water in a 400-mL PYREX® beaker with a medium-speed propeller (600 rpm) until a homogenous suspension was produced.

The Phase 2 ingredients were premixed in a 100-mL PYREX® beaker by stirring with a medium-speed propeller (300 rpm) for approximately 20-30 minutes until the CBD isolate was fully dissolved, thereby forming a cannabinoid suspension in the MCT carrier fluid. Then, the premixed Phase 2 suspension was added to the Phase 3 emulsifier in a 100-mL PYREX® beaker and mixed with a propeller mixer at 400 rpm for 20-30 minutes to produce a cannabinoid formulation. The cannabinoid formulation (Phase 2/3) was added to Phase 1 in the 400-mL PYREX® beaker with medium propeller mixing (400-600 rpm) until homogenous after which, the pH of the formulation was adjusted to between 5.5 and 6.0 by addition of the Phase 4 ingredient. Finally, the Phase 5 ingredient was added to the 400-mL PYREX® beaker with continued thorough mixing to produce the third cannabinoid formulation.

### Example 4: Preparation of a Cannabinoid Formulation Using a Method to Pre-Associate the Emulsifier with a CBD Isolate and Using Safflower Oil to Solubilize the CBD Isolate

A fourth cannabinoid formulation was prepared using a method to pre-associate a selected emulsifier with a selected CBD isolate using the ingredients and the percentages shown in the Table 5.

TABLE 5

| Phase | Ingredient | Percentage |
|---|---|---|
| 1 | DI water | 77.73 or 86.73 |
| 1 | SEPIMAXZEN ® | 1.25 |
| 2 | CBD Isolate | 1.00 |
| 2 | safflower oil | 9.00 |
| 3 | Emulsifier | 10.00 |
| | A: Safflower oleosomes 65% | 10.00 |
| | B: Almond oleosomes 60% | 10.00 |
| | C: Hemp oleosomes 70% | 10.00 |
| | D: Canola oleosomes 52% | 10.00 |
| | E: Polysorbate 20 100% | 1.00 |
| 4 | Sodium Hydroxide Solution (18%) | 0.02 |
| 5 | EUXYL ® PE9010 | 1.00 |
| | TOTAL | 100.00 |

Phase I ingredients were mixed by slowly dispersing SEPIMAX ZEN® in DI water in a 400-mL PYREX® beaker with a medium-speed propeller (600 rpm) until a homogenous suspension was produced.

The Phase 2 ingredients were premixed in a 100-mL PYREX® beaker by stirring with a medium-speed propeller (300 rpm) for approximately 20-30 minutes until the CBD isolate was fully dissolved, thereby forming a cannabinoid suspension in a safflower oil carrier fluid. Then, the premixed Phase 2 suspension was added to the Phase 3 ingredient in a 100-ml PYREX® beaker and mixed with a propeller mixer at 400 rpm for 20-30 minutes to form a cannabinoid formulation. The cannabinoid formulation (Phase 2/3) was added to Phase 1 in the 400-mL PYREX® beaker with medium propeller mixing (400-600 rpm) until homogenous after which, the pH was adjusted to between 5.5 to 6.0 by the addition of the Phase 4 ingredient. Finally, the Phase 5 ingredient was added to the 400-mL PYREX® beaker with continued thorough mixing to produce the fourth cannabinoid formulation.

### Example 5: Confocal Raman Spectroscopy

Confocal Raman Spectroscopy was performed with Horiba LabRam HR Evolution equipment using the following conditions: laser 488 nm (Melles Griot Ar+ Ion Laser), power 6-8 mW at sample, objective 50×/0.5 LWD Olympus, confocal hole 50-75 micron, grating 600 gr/mm, mapping performed with SWIFT™ mode (dwell time 0.5-1 sec per pixel) and using the Synapse Water Cooled BIDD Detector. A silicon calibration was performed at the beginning of each of the sets to correct the spectrometer. Samples from Example 3 (A-pre-association—safflower oleosome) and Example 2 (A-post-add—safflower oleosome) were pipetted onto separate glass cover slips and each was gently flattened with another fresh glass coverslip. The transmission lamp was aligned to help enhance optical contrast in the micrograph and the isolated components were tested to identify unique spectral regions for active product-CBD/MCT/oleosome/aqueous matrix. Maps were setup in a defined spectral region to capture unique features for all components within the sample. This range was 1380-3130 cm⁻¹ for all samples tested. Analyses of the Raman map data set were completed via component-least-square spectral loadings from the isolated specimen with both loading and results normalized. Scores for water, MCT, safflower oil, CBD, cover slip, and error for each of the samples were determined for each of the samples from Example 2 (A-post add—safflower oleosome) and Example 3 (A-pre-association—safflower oleosome).

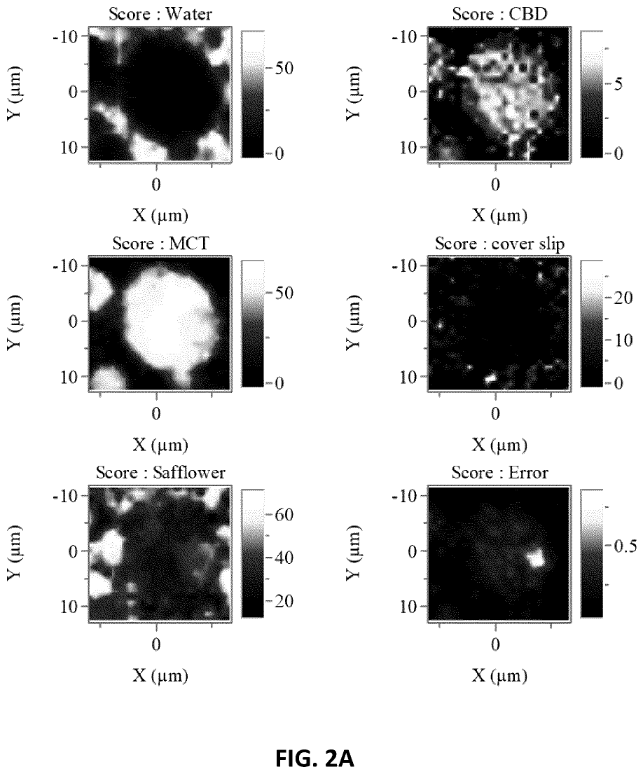

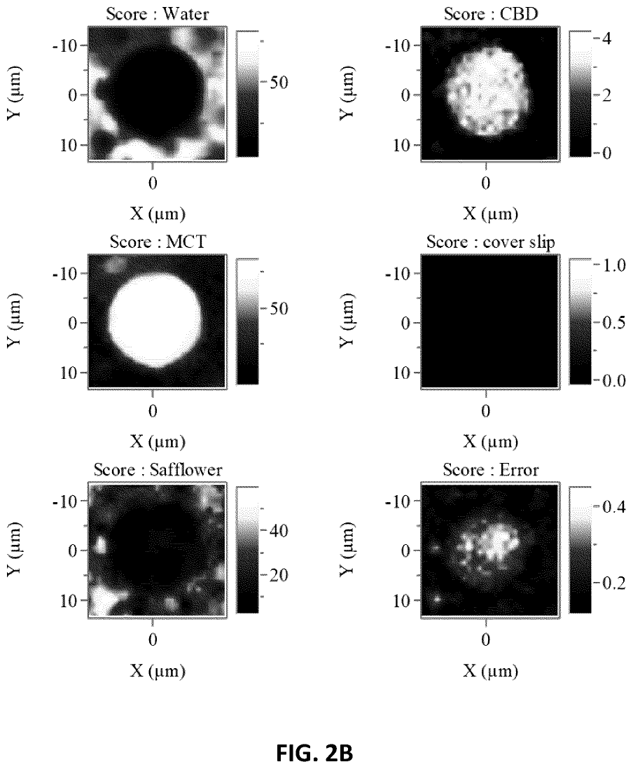

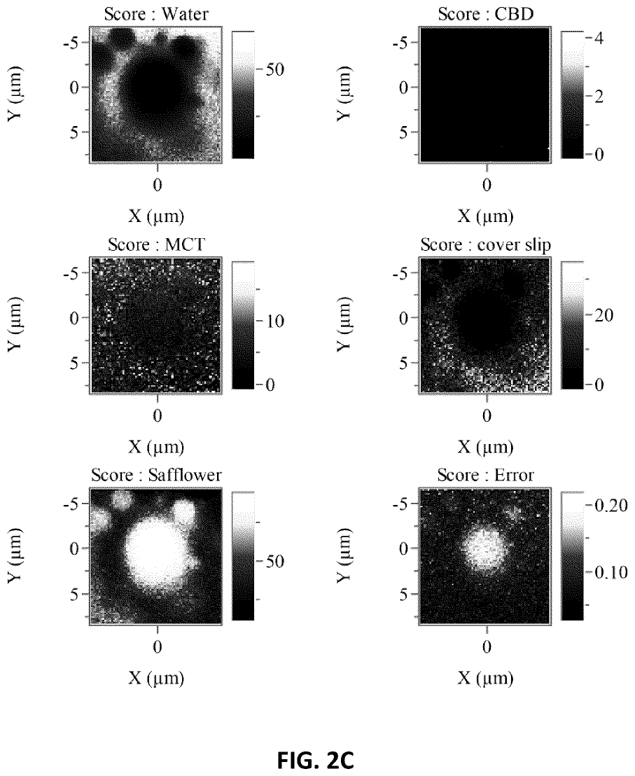

As shown in FIG. 2A (pre-association—safflower oleosomes sample 1) and in Table 6, the CBD, MCT, and safflower components were localized in the oleosome sphere (oleosome 1) with CLS scores of 8.0%, 66.7% and 25.0% respectively. As shown in Table 6, the CBD, MCT and safflower components were localized in the oleosome sphere (oleosome 2) with CLS scores of 3.9%, 23.1% and 64.2% respectively. As shown in FIG. 2B (pre-association—safflower oleosome sample 3) and Tables 6, the CBD, MCT, and safflower components were localized in the oleosome sphere (oleosome 3) with CLS scores of 3.5%, 77.4% and 13.8%, respectively. As shown in FIG. 2C (post-add—safflower oleosome) and Table 6, the CLS score for the CBD component was below the detectable limit, while the MCT and safflower components were localized in oleosome sphere with CLS scores of 12.0% and 81.7%, respectively.

TABLE 6

| | Pre-Association | Pre-Association | Pre-Association | Post-Add |
|---|---|---|---|---|
| | Safflower | Safflower | Safflower | Safflower |
| | Oleosome 1 | Oleosome 2 | Oleosome 3 | Oleosome |
| | (FIG. 2A) | (image not shown) | (FIG. 2B) | (FIG. 2C) |
| Component | CLS Score | CLS Score | CLS Score | CLS Score |
| CBD | 8.0% | 3.9% | 3.5% | ND |
| MCT | 66.7% | 23.1% | 77.4% | 12.0% |
| Safflower | 25.0% | 64.2% | 13.8% | 81.7% |

As shown FIG. 2B (pre-association—safflower oleosome sample 2) and Table 7, CBD was associated with the safflower oleosome, whereas in FIG. 2C (post-add—safflower oleosome) and Table 7, the level of CBD was below the detectable limit and was not associated with the safflower oleosome. The CBD appeared to be either inside of the lipid core of the oleosome and/or surrounding the protein/phospholipid membrane of the safflower oleosome in the pre-association sample, however it was not associated with the oleosome in the post-add sample.

TABLE 7

| | Pre-Association - | Post-Add - |
|---|---|---|
| | Safflower | Safflower |
| | Oleosome 2 | Oleosome |
| | (FIG. 2B) | (FIG. 2C) |
| Component | CLS Score | CLS Score |
| CBD associated with oleosome | 3.9% | ND |
| CBD inside or surrounding oleosome | 3.5% | ND |
| CBD outside oleosome | 0.0% | ND |
| CBD associated with water phase | 1.0% | ND |

These results suggest that (i) the CBD was associated with the oleosomes in formulations prepared with the pre-association method, but (ii) that the CBD was not associated with the oleosomes in formulations prepared with the post-add method. The MCT oil was the carrier molecule that was used for solubilisation of the CBD therein. It is also interesting to note that the MCT oil in the pre-associated sample (FIG. 2B, middle row, left hand column) appeared to be localized with the safflower oleosome but was more dispersed in the post-associated sample (FIG. 2C, middle row, left hand column).

### Example 6: Evaluation of CBD Concentration Using HPLC Analyses

A standard curve for cannabidiol (CBD) was generated using a Prominence-I HPLC system from Shimadzu equipped with a UV detector. A linear dynamic range was established at 0.5 to 100 mg/L (ppm) for the CBD. Gradient elution conditions with acid-modified water and acetonitrile were employed with a C18 column to achieve separation in under 10 minutes. Samples containing CBD were diluted using methanol to accurately quantify the CBD so that its response was within the linear range for CBD and the samples were subsequently shaken for 30 seconds. Each sample mixture was filtered through a 0.2 um PTFE syringe filter into a HPLC vial.

### Example 7: Evaluation of CBD Degradation by HPLC Analyses of Samples Stored at 45° C.

Samples were prepared following the methods set out in Example 2 (post-add, MCT oil to solubilize CBD), Example 3 (pre-association, MCT oil to solubilize CBD) and Example 4 (pre-association, safflower oil to solubilize CBD) using either safflower oleosomes, hemp oleosomes, Canola oleosomes, almond oleosomes, or polysorbate as the emulsifier. The samples were stored at 45° C. for a period of 2 months. It should be noted that the storage of such samples at 45° C. for 3 months is equivalent to storage of the samples at ambient room temperatures for about 2 years. Table 8 shows the effects of storage time on stability of the CBD concentrations in each of the samples, wherein the CBD concentrations were determined with the HPLC procedure outlined in Example 6 at Time 0, 1 month, and 2 months. It should be noted that that the starting concentrations of CBD at Time 0 are expressed as a reference number 1.0, and the losses of CBD over time are expressed as numerical fractions.

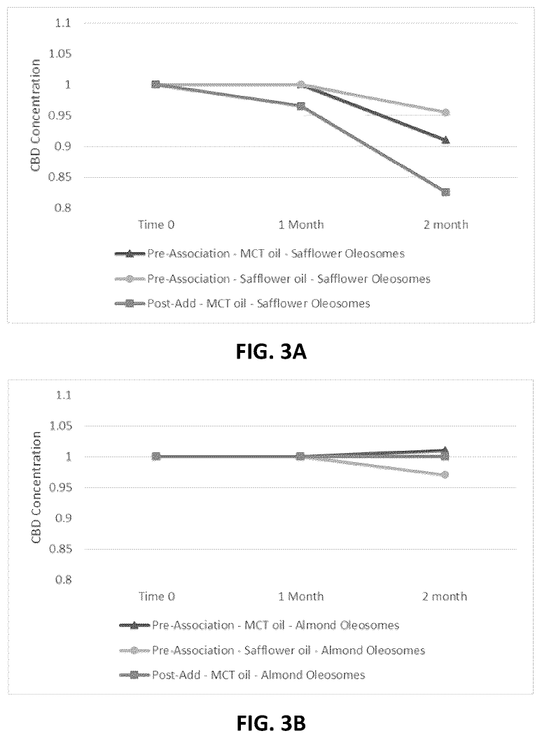

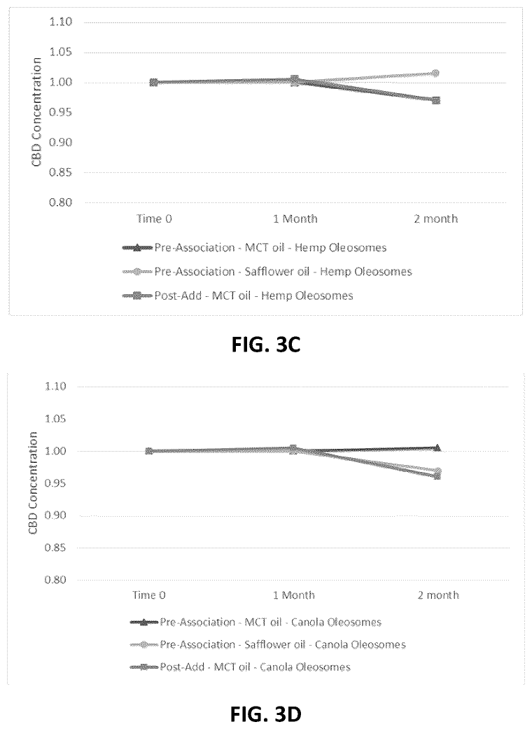

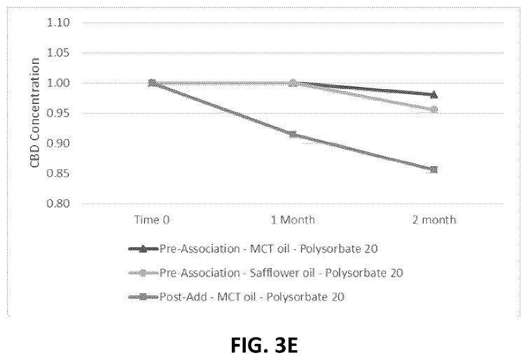

As shown in Table 8 and FIG. 3A, the CBD concentrations in samples prepared using safflower oleosomes, decreased over the 2-month storage period at 45° C. When comparing the post-add method of formulation with the pre-association method of formulation with either MCT oil or safflower oil as the CBD carrier, the CBD concentrations remained higher in pre-association methods with both MCT oil and the safflower oil as the CBD carrier. When the almond, hemp and Canola oleosomes were used as the emulsifier ingredients, the CBD concentrations remained relatively unchanged over the 2-month period at 45° C. storage (FIG. 3B; almond oleosomes), (FIG. 3C; hemp oleosomes), (FIG. 3D; Canola oleosomes). In regard to the hemp oleosomes (FIG. 3C) and the Canola oleosomes (FIG. 3D), the pre-association method of formulation provided more stable formulations with limited reductions in CBD concentration over time, and it was apparent that formulations prepared by the pre-association method were more stable than were the formulations prepared with the post-association method. When polysorbate 20 was as an emulsifier ingredient, the pre-association method with MCT oil as the CBD carrier provided more storage stability to the formulations that did the post-add method of formulation (FIG. 3E). These results demonstrate that the pre-association method for formulation wherein the CBD is combined with a selected oleosome, provides a more storage-stable CBD formulation than does the post-add method of formulation.

TABLE 8

| | CBD Concentration | | |
|---|---|---|---|
| Sample | Time 0 | 1 month | 2 months |
| Pre-Association-MCT oil-Safflower Oleosomes | 1.00 | 1.00 | 0.91 |
| Pre-Association-Safflower oil-Safflower Oleosomes | 1.00 | 1.00 | 0.96 |
| Post-Add-MCT oil-Safflower Oleosomes | 1.00 | 0.97 | 0.83 |
| Pre-Association-MCT oil-Almond Oleosomes | 1.00 | 1.00 | 1.01 |
| Pre-Association-Safflower oil-Almond Oleosomes | 1.00 | 1.00 | 0.97 |
| Post-Add-MCT oil-Almond Oleosomes | 1.00 | 1.00 | 1.00 |
| Pre-Association-MCT oil-Hemp Oleosomes | 1.00 | 1.00 | 0.97 |
| Pre-Association-Safflower oil-Hemp Oleosomes | 1.00 | 1.00 | 1.02 |
| Post-Add-MCT oil-Hemp Oleosomes | 1.00 | 1.01 | 0.97 |
| Pre-Association-MCT oil-Canola Oleosomes | 1.00 | 1.00 | 1.01 |
| Pre-Association-Safflower oil-Canola Oleosomes | 1.00 | 1.00 | 0.97 |
| Post-Add-MCT oil-Canola Oleosomes | 1.00 | 1.01 | 0.96 |
| Pre-Association - MCT oil - Polysorbate 20 | 1.00 | 1.00 | 0.98 |
| Pre-Association-Safflower oil-Polysorbate 20 | 1.00 | 1.00 | 0.96 |
| Post-Add-MCT oil-Polysorbate 20 | 1.00 | 0.92 | 0.86 |

### Example 8: Colorimetric Assessment of CBD Degradation

The difference in color between two samples may be obtained by measuring “how far away” one color is from another. The color difference may be determined by comparing the ΔE of two samples. As used herein, the term “ΔE” refers to a measure of change in visual perception of two colors that allows for quantified determination of the differences between two colors within a color space.

The formula for ΔE is:

ΔE=ΔL°²+Δa°²+Δb°²

ΔL°=L°_{Probe}−L°_{Ref}

Δa°=a°_{Probe}−a°_{Ref}

Δb°=b°_{Probe}−b°_{Ref} Eq. 1

L* is normalized to the values 0 to 100, corresponding to a percentage scale which describes the lightness of a sample. L*=100% means 100% light and L*=0 means no light (black). Positive a* values represent reddish tones and negative values greenish tones. A more positive a* value indicates the tone is more reddish. A more negative a* value indicates the tone is more greenish. Positive b* values represent yellowish tones and negative b* values represent blueish tones. A more positive b* value indicates the tone is more yellowish. A more negative b* value indicates the tone is more blueish. The color measurements of each sample were captured by a Spectro 1™ colorimeter device (available from Variable Inc., Chattanooga, Tenn., USA) by pointing the device at a container containing a sample and then recording the color of the target sample. The captured and recorded data were compared to the original color of each sample captured and recorded with the same device at Time 0, and the ΔE values were calculated from the comparisons.

### Example 9: Color-Change-Based Evaluations of CBD Degradation in Formulated Samples Stored at 45° C.

Samples of CBD formulations were prepared following the methods disclosed in Example 2 (post-add, MCT to solubilize CBD) and Example 3 (pre-association, MCT to solubilize CBD) using safflower oleosomes, hemp oleosomes, Canola oleosomes, almond oleosomes, or polysorbate 20 as the emulsifier ingredients. The samples were stored at 45° C. for a period of 2 months. The table below provides the colorimetric change, expressed as ΔE, for each of the samples using the procedure as outlined in Example 8. It should be noted that in these formulations, CBD turned pink as it degraded, and a greater extent of degradation resulted in a stronger, more intense pink color.

TABLE 9

| | ΔE |
|---|---|
| Sample | (Ref. vs 45° C.) |
| Pre-Association - MCT oil - Safflower Oleosomes | 22.06 |
| Post-Add - MCT oil - Safflower Oleosomes | 26.55 |
| Pre-Association - MCT oil - Almond Oleosomes | 12.34 |
| Post-Add - MCT oil - Almond Oleosomes | 12.38 |
| Pre-Association - MCT oil - Hemp Oleosomes | 7.98 |
| Post-Add - MCT oil - Hemp Oleosomes | 11.77 |
| Pre-Association - MCT oil - Canola Oleosomes | 4.22 |
| Post-Add - MCT oil - Canola Oleosomes | 6.77 |
| Pre-Association - MCT oil - Polysorbate 20 | 11.86 |
| Post-Add - MCT oil - Polysorbate 20 | 30.03 |

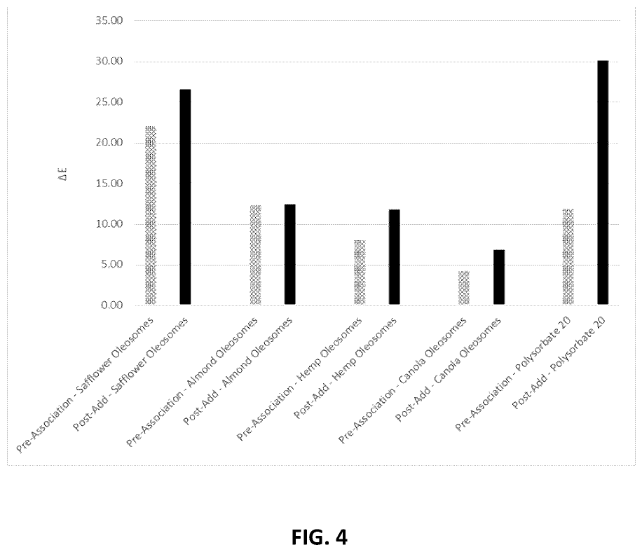

As shown in Table 9 and FIG. 4, there was a more significant color change indicated by a larger ΔE value, for all of the emulsifiers tested including safflower, almond, hemp, Canola oleosomes or polysorbate 20 when the post-add method of formulation was used. This data demonstrates that the pre-association formulation method wherein CBD is first associated with a selected oleosome and then further formulated, protects the CBD from degradation to a greater extent than does the post-add method of formulation.

### Example 10: Color-Change-Based Evaluations of CBD Degradation in Formulated Samples Stored Under UV Exposure Conditions

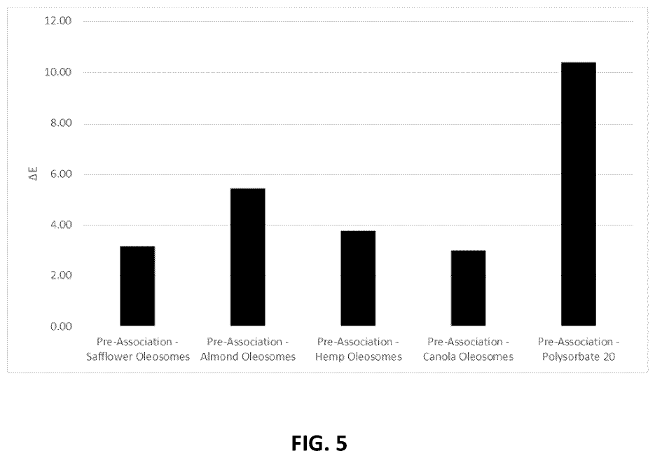

Samples of CBD formulations were prepared following the methods disclosed in Example 3 (pre-association, MCT to solubilize CBD) using safflower oleosomes, hemp oleosomes, Canola oleosomes, almond oleosomes, or polysorbate 20 as the emulsifier ingredients. The formulated samples were stored under UV conditions in ambient temperature conditions in a fan-cooled box positioned about 30 centimeters from UV light source for a period of 2 months (UV light at 540 KJ/hr or 150 Watts across a spectrum from UVA1 to Infrared light (340-700 nm). Table 10 and FIG. 5 show the color change data for the samples wherein the data was generated using the procedure outlined in Example 8. CBD turns pink as it degrades and the more degradation, the stronger the pink color is. All of the oleosome formulation samples exhibited less of a color difference over time when compared to the color differences for the samples wherein the polysorbate was the emulsifier.

TABLE 10

| | ΔE |
|---|---|
| Sample | (Ref. vs UV) |
| Pre-Association - MCT oil - Safflower Oleosomes | 3.15 |
| Pre-Association - MCT oil - Almond Oleosomes | 5.41 |
| Pre-Association - MCT oil - Hemp Oleosomes | 3.75 |
| Pre-Association - MCT oil - Canola Oleosomes | 2.97 |
| Pre-Association - MCT oil - Polysorbate 20 | 10.39 |

### Example 11: Preparation of Cannabinoid Formulations Having Different CBD:MCT Oil Ratios

The ratio of CBD:MCT oil in the method set out in Example 3 (pre-association) was 10:90. In order to determine optimal CBD:MCT oil ratios, samples containing ratios of 10:90, 25:75, and 35:65 were formulated. All of the compositions were produced following the method set out in Example 3 so that all of the compositions comprised 1% CBD oil in all cases (Table 11). Safflower oleosomes, hemp oleosomes, or polysorbate 20 were prepared at 10% composition with the water levels adjusted to an equivalence of 100%.

TABLE 12

| CBD | MCT oil | Parts for 1% | Total oil | Oleosome/MCT |
|---|---|---|---|---|
| (parts) | (parts) | CBD | (%) | ratio |
| 10 | 90 | 10 | 18.8 | 1.11 |
| 25 | 75 | 4 | 12.8 | 3.33 |
| 35 | 65 | 2.86 | 11.7 | 5.38 |

### Example 12: Color-Change-Based Evaluations of CBD Degradation in Formulations with Different Ratios of CBD:MCT Oil

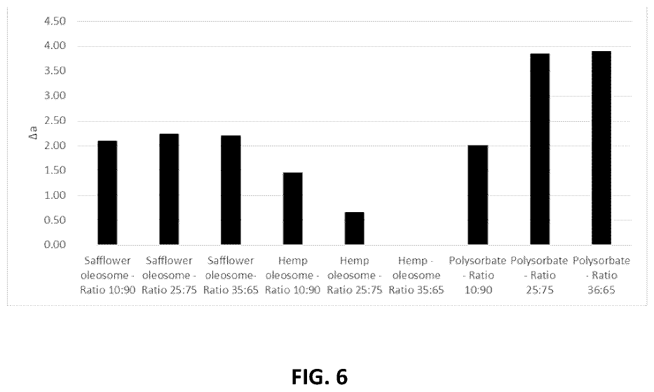

Samples of CBD formulations were prepared following the methods disclosed in Example 11 (pre-association, MCT to solubilize CBD) using safflower oleosomes, hemp oleosomes, Canola oleosomes, almond oleosomes, or polysorbate 20 as the emulsifier ingredients. The samples were stored under UV conditions (ambient temperature) for 2 weeks. The color of each sample was measured as set out in Example 8. The La data representing the red/green color shifts that occurred in the CBD:MCT oil ratios during the 2-week storage period, are shown in Table 12 and in FIG. 6. It should be noted that as the Aa values increase, the sample colors become more “reddish”.

The data for the CBD hemp oleosome formulations show that when less oil was present (i.e. in the 35:65 CBD:MCT ratio), the oleosomes protected CBD stability as indicated by the smaller color changes. This is in contrast to the CBD polysorbate formulations wherein as the level of oil in the ratio was decreased, the CBD increasingly degraded as shown by the larger color changes. The data also suggest that hemp oleosome formulations provided better protection against CBD degradation than was observed with the safflower formulations at all CBD:MCT ratios.

TABLE 12

| | | Δa |
|---|---|---|
| Formulation | CBD:MCT ratio | (Ref. vs UV) |
| Safflower oleosome | 10:90 | 2.10 |
| Safflower oleosome | 25:75 | 2.24 |
| Safflower oleosome | 35:65 | 2.20 |
| Hemp oleosome | 10:90 | 1.45 |
| Hemp oleosome | 25:75 | 0.65 |
| Hemp oleosome | 35:65 | 0.00 |
| Polysorbate | 10:90 | 2.00 |
| Polysorbate | 25:75 | 3.85 |
| Polysorbate | 36:75 | 3.90 |

1. A method of preparing a cannabinoid formulation comprising:
providing one or more cannabinoid compounds;
providing an oleosome suspension comprising a plurality of oleosomes; and
combining the oleosome suspension with the one or more cannabinoid compounds to form a cannabinoid formulation, wherein the one or more cannabinoid compounds is/are associated with the plurality of oleosomes in the oleosome suspension.

2. The method according to claim 1, additionally comprising a first step of preparing the oleosome suspension from a selected plant material.

3. The method according to claim 1, wherein the oleosome suspension is buffered.

4. The method according to claim 1, wherein the one or more cannabinoid compounds is at least one of cannabidiol (CBD), Δ⁹-tetrahydrocannabinol (Δ⁹-THC), cannabichromene (CBC), and mixtures thereof.

5. The method according to claim 1, wherein the one or more cannabinoid compounds is one or more substantially pure compounds.

6. The method according to claim 1, wherein the one or more cannabinoid compounds is a cannabinoid suspension consisting substantially of one or more cannabinoid compounds and a carrier fluid.

7. The method according to claim 6, wherein the carrier fluid is a plant oil.

8. The method according to claim 6, wherein the carrier fluid is a medium-chain triglyceride oil.

9. The method according to claim 6, wherein the cannabinoid suspension comprises from about 5% (w/w) to about 40% (w/w) of the one or more cannabinoid compounds.

10. The method according to claim 6, wherein the cannabinoid suspension comprises about 10% (w/w) of the one or more cannabinoid compounds.

11. The method according to claim 1, wherein the plurality of oleosomes of the oleosome suspension are recovered from a plant material selected from Carthamus plants, Prunus plants, Cannabis plants, or Brassica plants.

12. The method according to claim 1, wherein the cannabinoid formulation comprises the oleosome suspension, the one or more cannabinoid compounds, and water, which together constitute at least 90% (w/w), at least 95% (w/w), at least 96% (w/w), at least 97% (w/w), at least 98% (w/w), or at least 99% (w/w) of the cannabinoid formulation.

13. The method according to claim 6, wherein the cannabinoid suspension additionally comprises water and together, constitutes at least 90% (w/w), at least 95% (w/w), at least 96% (w/w), at least 97% (w/w), at least 98% (w/w), or at least 99% (w/w) of the cannabinoid formulation.

14. A method of preparing a consumer product comprising one or more cannabinoid compounds, the method comprising:
providing a cannabinoid formulation comprising one or more cannabinoid compounds and an oleosome suspension comprising a plurality of oleosomes, wherein the one or more cannabinoid compounds is/are associated with the plurality of oleosomes; and
formulating the cannabinoid formulation with at least one additional ingredient suitable for the preparation of a consumer product.

15. The method according to claim 14, wherein the at least one additional ingredient is a diluent, an excipient, a carrier, or mixtures thereof.

16. The method according to claim 15, wherein the diluent is an aqueous solution.

17. The method according to claim 14, additionally comprising one or more of a preservative, an antimicrobial, and an antioxidant.

18. The method according to claim 14, wherein the consumer product contains from about 0.1% (v/v) to about 10% (v/v) of the cannabinoid formulation.

19. The method according to claim 14, wherein the oleosome suspension is a buffered suspension.

20. The method according to claim 14, wherein the plurality of oleosomes of the oleosome suspension are recovered from Carthamus plants, Prunus plants, Cannabis plants, or Brassica plants.

21. The method according to claim 14, wherein the one or more cannabinoid compounds is at least one of cannabidiol (CBD), Δ⁹-tetrahydrocannabinol (Δ⁹-THC), cannabichromene (CBC), or mixtures thereof.

22. The method according to claim 14, wherein the consumer product is a personal care product.

23. The method according to claim 14, wherein the consumer product is a food product.

24. The method according claim 14, wherein the consumer product is a pharmaceutical product.

25. The method according to claim 14, wherein the consumer product is a nutraceutical product.

26. The method according to claim 14, wherein the consumer product comprises from about 0.1% (w/w) to about 10% (w/w) of the cannabinoid compound.

27. A method of preparing a consumer product comprising one or more cannabinoid compounds, the method comprising:
providing one or more cannabinoid compounds;
providing an oleosome suspension comprising a plurality of oleosomes;
combining the oleosome suspension with the one or more cannabinoid compounds to form a cannabinoid formulation, wherein the one or more cannabinoid compounds is/are associated with the plurality of oleosomes; and
formulating the cannabinoid formulation with at least one additional ingredient suitable for a consumer product.

28. The method according to claim 27, additionally comprising a first step of preparing the oleosome suspension from a selected plant material.

29. The method according to claim 27 wherein the at least one additional ingredient is a diluent, an excipient, a carrier, or mixtures thereof

30. The method according to claim 29, wherein the diluent is an aqueous solution.

31. The method according to claim 27, additionally comprising one or more of a preservative, an antimicrobial, and an antioxidant.

32. The method according to claim 27, wherein the consumer product contains from about 0.1% (v/v) to about 10% (v/v) of the cannabinoid formulation.

33. The method according to claim 27, wherein the oleosome suspension is a buffered suspension.

34. The method according to claim 27, wherein the plurality of oleosomes of the oleosome suspension are recovered from Carthamus plants, Prunus plants, Cannabis plants, or Brassica plants.

35. The method according to claim 27, wherein the one or more cannabinoid compounds is at least cannabidiol (CBD), Δ⁹-tetrahydrocannabinol (Δ⁹-THC), cannabichromene (CBC), or mixtures thereof.

36. The method according to claim 27, wherein the consumer product is a personal care product.

37. The method according to claim 27, wherein the consumer product is a food product.

38. The method according to claim 27, wherein the consumer product is a nutraceutical product.

39. The method according to claim 27, wherein the consumer product is a pharmaceutical product.

40. The method according to claim 27, wherein the consumer product comprises from about 0.1% (w/w) to about 10% (w/w) of the cannabinoid compound.

41. A cannabinoid formulation comprising:
one or more cannabinoid compounds;
an oleosome suspension comprising a plurality of oleosomes, wherein the one or more cannabinoid compounds is/are associated with the plurality of oleosomes; and
optionally additional water.

42. A cannabinoid formulation according to claim 41, wherein the one or more cannabinoid compounds are provided in a cannabinoid suspension wherein the cannabinoid suspension comprises the one or more cannabinoid compounds and a carrier fluid.

43. A cannabinoid formulation according to claim 42, wherein the carrier fluid is a plant oil.

44. A cannabinoid formulation according to claim 42, wherein the carrier fluid is a medium-chain triglyceride oil.

45. A cannabinoid formulation according to claim 41, wherein the cannabinoid suspension comprises from about 5% (w/w) to about 40% (w/w) of the cannabinoid compound.

46. A cannabinoid formulation according to claim 41, wherein the cannabinoid suspension comprises about 10% (w/w) of the cannabinoid compound.

47. A cannabinoid formulation according to claim 41, wherein the plurality of oleosomes of the oleosome suspension are recovered from Carthamus plants, Prunus plants, Cannabis plants, or Brassica plants.

48. A cannabinoid formulation according to claim 41, wherein the one or more cannabinoid compounds is at least one of cannabidiol (CBD), Δ⁹-tetrahydrocannabinol (Δ⁹-THC), cannabichromene (CBC), or mixtures thereof.

49. A consumer product comprising a cannabinoid formulation, the cannabinoid formulation comprising:
one or more cannabinoid compounds;
an oleosome suspension comprising a plurality of oleosomes, wherein the one or more cannabinoid compounds is/are associated with the plurality of oleosomes; and
at least one additional ingredient.

50. A consumer product according to claim 49, wherein the at least one additional ingredient is a diluent, an excipient, a carrier, or mixtures thereof.

51. A consumer product according to claim 49, wherein the at least one additional ingredient one or more of a preservative, an antimicrobial, an antioxidant, or mixtures thereof.

52. A use of a cannabinoid formulation comprising one or more cannabinoid compounds and an oleosome suspension comprising a plurality of oleosomes, wherein the one or more cannabinoid compounds is/are associated with the plurality of oleosomes to prepare a consumer product.
